(19) United States
(12) Patent Application Publication
(10) Pub. No.: US 2022/0399076 A1
(43) Pub. Date: Dec. 15, 2022

(54) METHOD OF IDENTIFYING CANDIDATE GENE FOR GENETIC DISEASE

(71) Applicant: AJOU UNIVERSITY INDUSTRY-ACADEMIC COOPERATION FOUNDATION, Suwon-si (KR)

(72) Inventors: Hyun Jung SHIN, Gyeonggi-do (KR); Dong Gi LEE, Gyeonggi-do (KR); Sang Joon SON, Seoul (KR); Chang Hyung HONG, Gyeonggi-do (KR)

(73) Assignee: AJOU UNIVERSITY INDUSTRY-ACADEMICCOOPERATION FOUNDATION, Suwon-si (KR)

(21) Appl. No.: 17/513,001

(22) Filed: Oct. 28, 2021

(30) Foreign Application Priority Data
Jun. 9, 2021 (KR) 10-2021-0074803

(51) Int. Cl. G16B 20/20; G16B 40/30; G06N 20/00; G06N 5/02

(52) U.S. Cl. CPC G16B 20/20; G16B 40/30; G06N 20/00; G06N 5/027

(57) ABSTRACT

Provided is a method of identifying a candidate gene for a genetic disease includes obtaining a disease network, disease-gene association information, and a gene network, obtaining a single nucleotide polymorphism (SNP) network based on intra-relation data between a plurality of SNPs, and inter-relation data between genes and SNPs, creating a disease-gene-SNP multilayered network based on the disease network, the disease-gene association information, the gene network, the SNP network, and the interrelation data between genes and SNPs, and identifying a candidate gene for a genetic disease using the multilayered network.

### CROSS-REFERENCE TO RELATED APPLICATION

This application claims the benefit of Korean Patent Application No. 10-2021-0074803, filed on Jun. 9, 2021, in the Korean Intellectual Property Office, the disclosures of which are incorporated herein in its entirety by reference.

### BACKGROUND

### 1. Field

The disclosure relates to a method of identifying a candidate gene causing a genetic disease such as dementia.

Example embodiments of the present disclosure relate to two national research and development projects. Information on one national research and development project has subject identification No. 1711133622, subject No. 2021R1A2C2003474, project name “Basic Science Research Program (Ministry of Science and ICT)”, and subject title “Artificial intelligence model development for dementia precision medicine and spectral diagnosis”. Information on the other national research and development project has subject identification No. 1465033894, subject No. KBN4B02, project name “Korea Biobank Project”, and subject title “Biobank Innovations for chronic Cerebrovascular disease With ALZheimer's disease Study (BICWALZS)”.

### 2. Description of the Related Art

Various approaches for diagnosing and treating genetic diseases such as dementia, especially for identifying core genes that affect an attack of a disease receive considerable attention these days. When a variation of the core gene can be tracked, it will be a great help to predict a time of the attack of the genetic disease and develop drugs for treatment.

However, discovering the core gene requires huge costs, time and efforts. To solve this problem, a study to improve efficiency of discovering a candidate gene using, for example, computational biology is actively being conducted.

### SUMMARY

The disclosure provides a method of effectively identifying candidate genes other than those known to be related to a genetic disease based on a layered network configured by reflecting single nucleotide polymorphism (SNP) data.

According to an aspect of the disclosure, a method of identifying a candidate gene for a genetic disease performed by a computer-executable program, the method comprising: obtaining a disease network, disease-gene association information, and a gene network; obtaining a single nucleotide polymorphism (SNP) network based on intra-relation data between a plurality of SNPs, and inter-relation data between genes and SNPs; creating a disease-gene-SNP layered network based on the disease network, the disease-gene association information, the gene network, the SNP network, and the interrelation data between genes and SNPs; and identifying a candidate gene for a genetic disease using the layered network.

According to an exemplary embodiment, the SNP network comprises a plurality of nodes each corresponding to an SNP type and at least one edge representing connection between the plurality of nodes, and wherein each of the at least one edge represents a degree of similarity based on the intra-relation between the connected SNPs.

According to an exemplary embodiment, each of the at least one edge has a value obtained based on odds ratios in a logistic regression model based on allele dosage of the connected SNPs.

According to an exemplary embodiment, the creating of the layered network comprises setting values of gene-SNP edges between the gene network and the SNP network based on the interrelation data between genes and SNPs, and wherein the setting of the values of gene-SNP edges comprises: when a first SNP corresponding to a first node among nodes of the SNP network belongs to a first gene corresponding to a second node among nodes of the gene network, setting a value of a gene-SNP edge between the first node and the second node to 1; and when the first SNP does not belong to the first gene, setting the value of the gene-SNP edge between the first node and the second node to 0.

According to an exemplary embodiment, the identifying of the candidate gene for the genetic disease using the layered network comprises: setting a label of nodes corresponding to genes and SNPs already known to be related to the genetic disease among nodes of the layered network to 1 and a label of the other nodes to 0; calculating a score for each of the genes using graph-based semi-supervised learning (SSL); and identifying a candidate gene for the genetic disease based on the calculated score.

According to an exemplary embodiment, the identifying of the candidate gene for the genetic disease based on the calculated score comprises identifying at least one gene with the calculated score higher than a reference score as the candidate gene.

According to an exemplary embodiment, the disease network comprises: a plurality of nodes each corresponding to a disease; and at least one edge representing connection between the plurality of nodes, wherein each of the at least one edge represents a degree of association between the connected diseases, and wherein the degree of association is calculated based on a number or a rate of genes common between the connected diseases.

According to an exemplary embodiment, the gene network comprises: a plurality of nodes each corresponding to a gene; and at least one edge representing connection between the plurality of nodes, wherein each of the at least one edge represents a degree of association between the connected genes, and wherein the degree of association is obtained from a database in which pieces of protein-protein interaction information are integrated.

According to an exemplary embodiment, the disease-gene association information comprises information about genes related to causing each disease, wherein the creating of the layered network comprises setting values of disease-gene edges between the disease network and the gene network based on the disease-gene association information, and wherein the setting of the values of disease-gene edges comprises: when a first gene corresponding to a first node among nodes of the gene network is identified based on the disease-gene association information as being related to causing a first disease corresponding to a second node among nodes of the disease network, setting a value of a disease-gene edge between the first node and the second node to 1; and when the first gene is not identified as being related to causing the first disease, setting the value of the disease-gene edge between the first node and the second node to 0.

According to an aspect of the disclosure, a method of identifying a candidate gene for a genetic disease performed by a computer-executable program, the method comprising: creating a single nucleotide polymorphism (SNP) network based on intra-relation data between a plurality of SNPs; creating a gene-SNP layered network based on a gene network, the SNP network, and intra-relation data between SNPs; and identifying a candidate gene for a genetic disease using the layered network.

### BRIEF DESCRIPTION OF THE DRAWINGS

The above and other features and advantages of the disclosure will become more apparent by describing in detail exemplary embodiments thereof with reference to the accompanying drawings in which:

FIG. 1 is a flowchart for describing a method of identifying a candidate gene for a genetic disease, according to an embodiment of the disclosure;

FIG. 2 illustrates a layered network configured to include a disease network and a gene network, according to an embodiment of the disclosure;

FIG. 3 illustrates a single nucleotide polymorphism (SNP) network, according to an embodiment of the disclosure;

FIG. 4 illustrates a disease-gene-SNP layered network formed by connecting the networks shown in FIGS. 2 and 3;

FIG. 5 illustrates matrices representing intra-relations between nodes of the same network and inter-relations between nodes of different networks in the layered network of FIG. 4;

FIGS. 6 and 7 are diagrams for describing operation of identifying candidate genes for genetic diseases using a disease-gene-SNP layered network configured according to an embodiment of the disclosure; and

FIG. 8 illustrates a candidate gene identification result using the disease-gene-SNP layered network according to an embodiment of the disclosure and a candidate gene identification result using a disease-gene layered network for comparison.

### DETAILED DESCRIPTION

Embodiments of the disclosure are provided to more fully explain the technical idea of the disclosure for those of ordinary skill in the art. The scope of the disclosure is not limited to the following embodiments of the disclosure, and various forms of modification can be made to the following embodiments of the disclosure without departing from the scope of the disclosure. The embodiments are provided to make the disclosure fully and completely understood and to fully convey the technical idea of the disclosure to those of ordinary skill in the art.

In the disclosure, the terms first, second, etc., are used to describe various members, areas, layers, portions and/or components, but it is obvious that the members, areas, layers, portions and/or components should not be limited by the terms. The terms do not imply a specific order, ranks or priorities, but are used to distinguish a member, area, portion, or component from another. Accordingly, a first member, area, portion, or component may also be referred to as a second member, area, portion, or component, without departing from the teachings of the disclosure. For example, a first component may be termed a second component, and a second component may be termed a first component, without departing from the teachings of the disclosure.

Unless otherwise defined, all the terms as herein used have the same meanings as those commonly understood by the ordinary skill in the art to which the disclosure pertains, including technical and scientific terms. Furthermore, the terms as commonly used and defined in the dictionary should be construed as having the same meaning as they imply in the context of relevant technologies, and unless explicitly defined, should not be interpreted in an overly formal sense.

Moreover, embodiments of the disclosure should not be limited to specific forms as illustrated in the accompanying drawings. For example, embodiments of the disclosure encompass changes of forms caused in a manufacturing process. In the drawings, like reference numerals indicate like elements, so overlapping description thereof will not be repeated.

The expression “A and/or B” as herein used includes each of A and B and both A and B.

Embodiments of the disclosure will now be described in detail with reference to accompanying drawings.

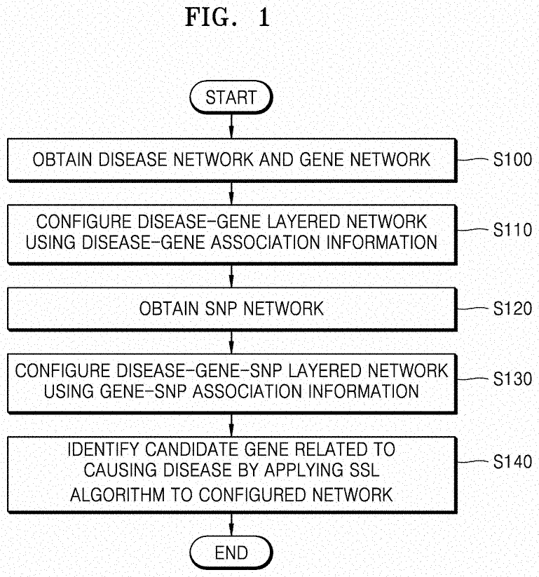

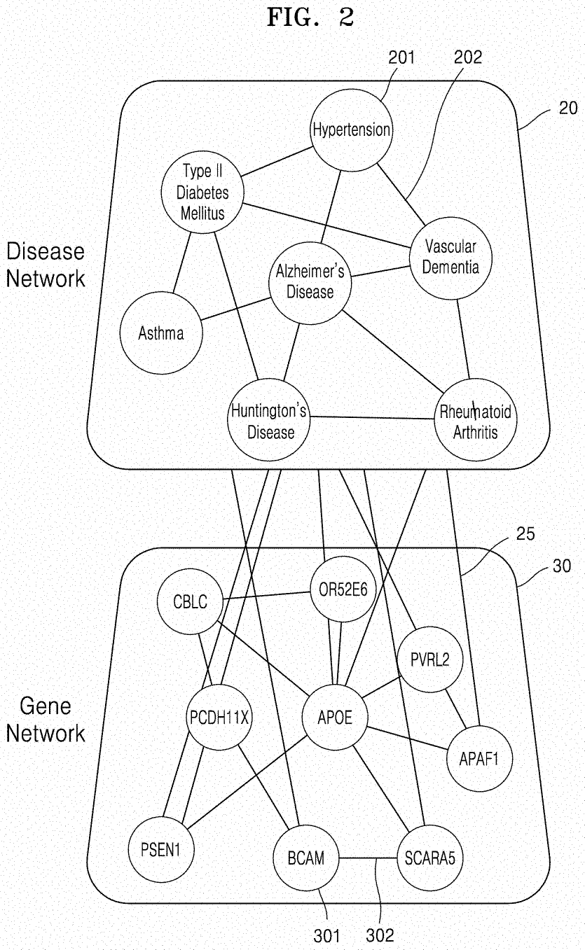

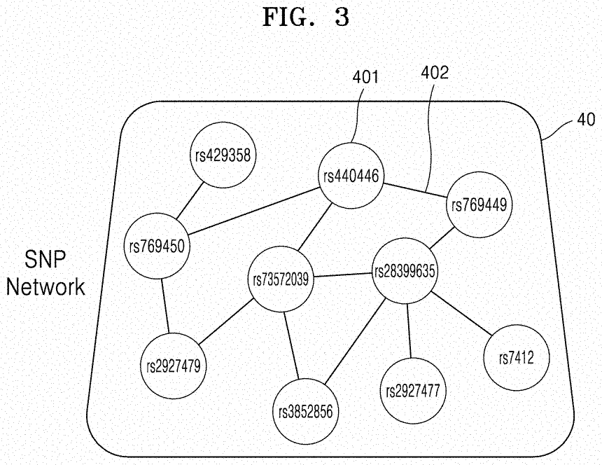

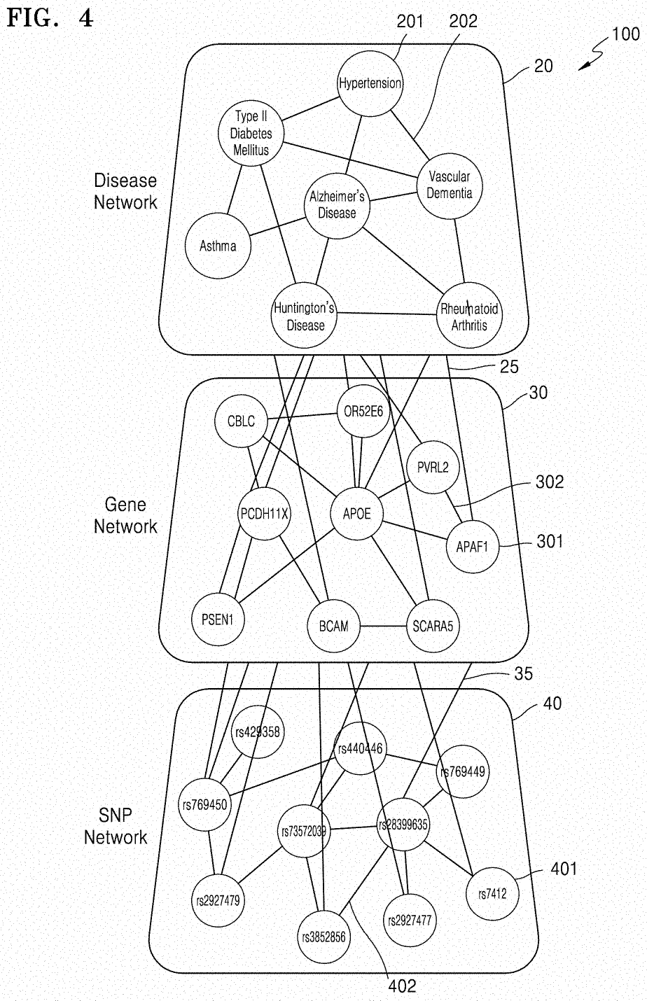

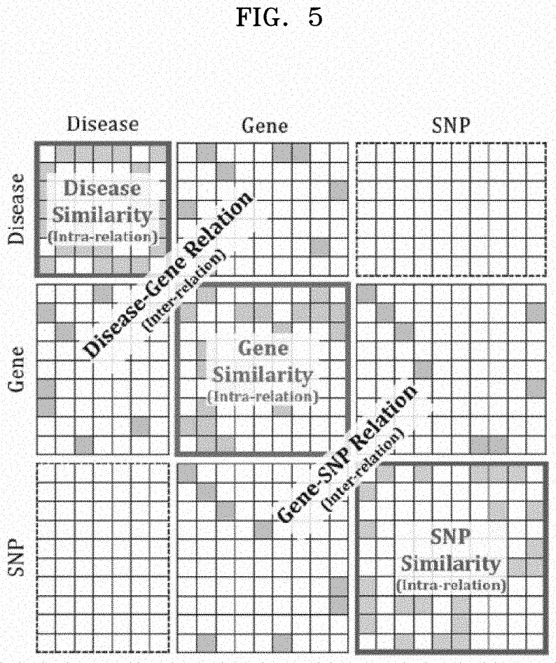

FIG. 1 is a flowchart for describing a method of identifying a candidate gene for a genetic disease, according to an embodiment of the disclosure. FIG. 2 illustrates a layered network configured to include a disease network and a gene network, according to an embodiment of the disclosure. FIG. 3 illustrates a single nucleotide polymorphism (SNP) network, according to an embodiment of the disclosure. FIG. 4 illustrates a disease-gene-SNP layered network formed by connecting the networks shown in FIGS. 2 and 3. FIG. 5 illustrates matrices representing intra-relations between nodes of the same network and inter-relations between nodes of different networks in the layered network of FIG. 4.

Embodiments of the disclosure may be applied to diseases related to genetic factors, i.e., genetic diseases (or genetic disorders). An attack of such a genetic disease may be caused by abnormalities of a specific protein due to a mutation of a specific gene. Hence, identifying a core gene related to the specific genetic disease and tracking a change of the identified gene may significantly help early prediction of the specific genetic disease and development of drugs for treatment.

Various embodiments of the disclosure will now be described by taking dementia as an example of the genetic disease, without being limited thereto.

Referring to FIG. 1, a method of identifying a candidate gene of a genetic disease according to an embodiment of the disclosure may include obtaining a disease network and a gene network in step S100, and configuring a disease-gene layered network using disease-gene association information in step S110.

Also referring to FIG. 2, a disease network 20 may include a plurality of nodes 201 each representing a specific disease and at least one edge 202 representing connection between the nodes.

For example, the plurality of nodes 201 are diseases (disorders) included in the dementia, which may correspond to Alzheimer's disease, Huntington's disease, vascular dementia, and the like. In some embodiments of the disclosure, the plurality of nodes 201 may further include nodes corresponding to various diseases other than the dementia.

The edge 202 connecting between the different nodes 201 may refer to a degree of association (e.g., similarity) between diseases corresponding to the different nodes 201. For example, the degree of association may have a value (weight) ranging from 0 to 1, and the higher the degree of association or similarity between diseases, the higher the value.

Specifically, the degree of association may be calculated based on a disease-gene relation. The disease-gene relation may indicate what genes are related to each disease. Taking this into account, the degree of association between a first disease and a second disease may be higher the more the number (or rate) of common genes between genes related to the first disease and genes related to the second disease. For example, the degree of association between two diseases may be calculated into the form of cosine similarity of disease vectors as in the following equation 1:

$${𝓌}_{ij} = \frac{{x}_{i} \cdot {x}_{j}}{‖ {x}_{i} ‖ \cdot ‖ {x}_{j} ‖} \qquad ( 1 )$$

where [symbol] ᵢⱼ denotes the degree of association between two diseases, Xᵢ and Xⱼ denote a first disease vector and a second disease vector, respectively, and Xᵢ·Xⱼ denotes an inner product of the first disease vector and the second disease vector.

As shown in FIG. 2, a gene network 30 may include a plurality of nodes 301 each representing a specific gene and at least one edge 302 representing connection between the nodes. The edge 302 connecting between the different nodes 301 may refer to a degree of association between diseases corresponding to the different nodes 301.

For example, the gene network 30 may be implemented based on a search tool for the retrieval of interacting genes/proteins (STRING) database (Szklarczyk et al., The STRING database in 2017: quality-controlled protein-protein association networks, made broadly accessible. Nucleic Acids Res., 45, D362-D368). The STRING database is a database that collects and integrates all of publicly available protein-protein interaction (PPI) information, which provides information encompassing almost all genes that amount to about 25,000 genes. The STRING database may include the degree of association between two different proteins, which is scored based on information such as a conserved gene neighborhood, gene fusion, phylogenetic co-occurrence, co-expression, experimental/biochemical data, literature co-occurrence, etc. The edge 302 of the gene network 30 may be obtained based on the degree of association scored based on each of the aforementioned information, and may be expressed as in the following equation 2:

S=1−Πᵢ(1−sᵢ) (2)

where sᵢ denotes the degree of association scored for each information, and i may correspond to the information. Value S calculated according to the equation 2 may correspond to a value of the edge 302.

Continuing to refer to FIG. 2, the disease network 20 and the gene network 30 may be connected by disease-gene edges 25 based on disease-gene association information to form a disease-gene layered network. The disease-gene association information includes information about genes that have influence on a specific disease, and may be provided according to data obtained previously in various methods. For example, when the disease-gene association information includes information indicating that ‘APOE’ gene influences Alzheimer's disease, the disease-gene edge 25 connecting a node corresponding to the Alzheimer's disease to a node corresponding to the ‘APOE’ gene may have a value of 1. On the other hand, when the disease-gene association information does not include information indicating that the specific gene influences the specific disease, the disease-gene edge 25 connecting between a node corresponding to the specific disease and a node corresponding to the specific gene may have a value of 0. In the embodiment of FIG. 2, shown is the disease-gene edge 25 having only the value of 1, for convenience of explanation.

Reference is made to FIG. 1 again.

The method may include obtaining a single nucleotide polymorphism (SNP) network in step S120, and configuring a disease-gene-SNP layered network using the gene-SNP association information in step S130.

The SNP refers to one or more nucleotide variants representing an individual difference in the DNA sequence. The SNP may represent individual genetic diversity, but in some cases a genetic disease may be caused by an SNP generated by a specific gene.

Most genetic studies related to genetic diseases such as dementia have so far selected specific SNPs representing specificity from among a patient group, and have been conducted by genome-wide association studies (GWAS) for targeting genes including the selected SNPs. In the GWAS, a gene related to a specific disease could be discovered by comparing genotypes of SNPs between a patient group and a control group and mapping markers related to the specific disease.

In the GWAS normally performed, however, there is a limit in that an influence of a single SNP identified by statistical analysis does not reflect complicated interactions made in a living body. It is hard to find a meaningful statistical difference between the patient group and the control group where there are individual SNPs, but when several SNPs are considered together, unexpected significant biological effects may occur.

Furthermore, an SNP may interact with a gene. For example, as the SNP occurs at a specific position in the specific gene, it may have effect on the structure or function of the gene, thereby causing a genetic disease. Hence, to identify a core gene for a disease, there is a need to consider not only the disease-gene relations but also relations between genes and SNPs.

Accordingly, in an embodiment of the disclosure, a method of identifying a candidate gene for a genetic disease may include considering intra-relations between SNPs and inter-relations between genes and SNPs together to identify candidate genes for a disease more effectively.

Based on this, referring to FIG. 3, an SNP network 40 may include a plurality of nodes 401 each corresponding to an SNP type and at least one edge 402 representing connection between the nodes. The edge 402 may refer to similarity (or a degree of similarity) based on intra-relations between different SNPs.

In an embodiment of the disclosure, the edge 402 may be calculated based on an epistasis test. The epistasis test may be performed using a logistic regression model as in the following equation 3 that classifies a patient group and a control group based on the allele dosage of SNP A and SNP B.

$$log \frac{Pr ( D = 1 \vert A , B )}{Pr ( D = 0 \vert A , B )} = {β}_{0} + {β}_{1} A + {β}_{2} B + {β}_{3} AB + e \qquad ( 3 )$$

where D may indicate a patient group when the value is 1 and a control group when the value is 0. In the epistasis test, the intra-relation (interaction) between SNPs may be calculated based on odds ratios from the logistic regression model. Especially, the intra-relation may be based on coefficient β₃ of the equation 3. When the odds ratio of the epistasis test has a value of 1, it may mean that there is no intra-relation between the two SNPs, and when the odds ratio grows away from the value of 1, it may mean that the intra-relation between the two SNPs increases. Taking this into account, a transformation function T(x) according to the following equation 4 is applied to the odds ratio, so that the value of the odds ratio is converted into a degree of similarity, and the converted value may correspond to a value (weight) of the edge 402. However, the method of determining a value of the edge 402 is not limited thereto, and various methods that reflect similarity and intra-relations between SNPs may be applied.

$$T ( x ) = \frac{2}{1 + {e}^{- inx}} - 1 \qquad ( 4 )$$

The SNP network 40 may be connected to the gene network 30 based on gene-SNP association information, and thus, a disease-gene-SNP layered network 100 may be configured as shown in FIG. 4. The gene-SNP association information is obtained from data of the patient group and/or the control group, and may include information about which gene each of the SNPs belongs to. For example, when ‘rs7412’ SNP belongs to ‘APOE’ gene, the gene-SNP edge 35 between the SNP and the gene may have a value of 1. On the other hand, when the ‘rs7412’ SNP does not belong to ‘APAF1’ gene, the gene-SNP edge 35 between the SNP and the gene may have a value of 0.

In the embodiment of the disclosure, it has been described that after configuration of the disease-gene layered network, the SNP network is combined thereto to form a disease-gene-SNP layered network. However, it will be appreciated by those of ordinary skill in the art that in some embodiments of the disclosure, after each of the disease network, the gene network, and the SNP network is obtained, the disease-gene-SNP layered network may be configured, and besides, the disease-gene-SNP layered network may be configured in other various orders.

Furthermore, although in FIGS. 2 to 4, only a certain number of nodes and edges are shown in each network for convenience of explanation, there may be huge number of nodes and edges in a network implemented in practice.

In the meantime, referring to FIG. 5, a pseudo matrix of the configured disease-gene-SNP layered network 100 may be provided. The pseudo matrix may include intra-matrices and inter-matrices. A disease-disease intra-matrix may represent a degree of association between diseases, and a disease-gene inter-matrix or a gene-disease inter-matrix may represent inter-relations between diseases and genes. A gene-gene intra-matrix may represent a degree of association between genes, and a gene-SNP inter-matrix or an SNP-gene inter-matrix may represent inter-relations between genes and SNPs. An SNP-SNP intra-matrix may represent intra-relations between SNPs.

Reference is made to FIG. 1 again.

The aforementioned method may include identifying candidate genes related to causing a disease by applying semi-supervised learning (SSL) to the configured layered network in step S140.

The SSL refers to a learning method having a combination of supervised learning and non-supervised learning. The SSL may use both labeled data and unlabeled data, thereby maximizing learning effects while minimizing an increase in time and cost required for labeling tasks.

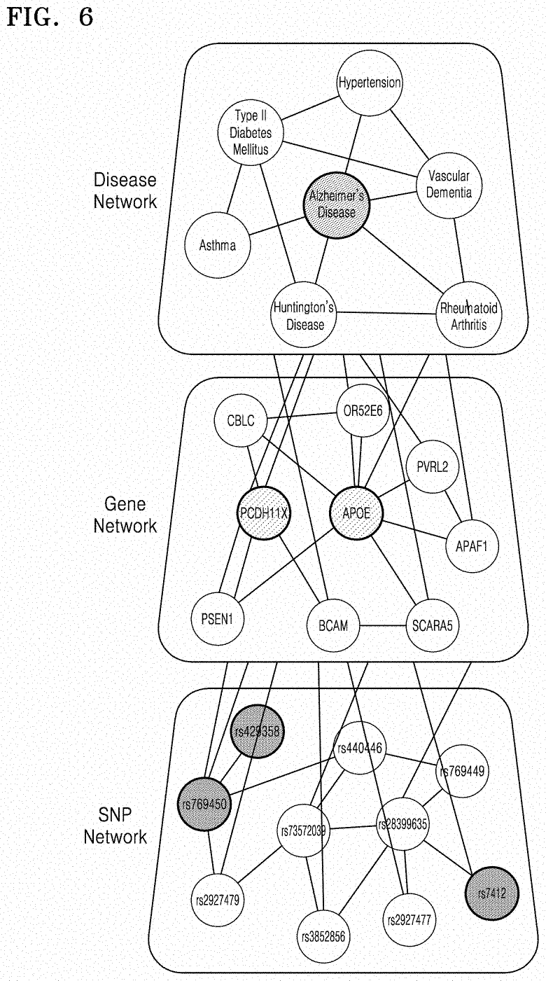

According to an embodiment of the disclosure, candidate genes related to causing a disease may be identified more effectively by applying the SSL to the disease-gene-SNP layered network configured as described above. This will be described below in detail in connection with FIGS. 6 to 8.

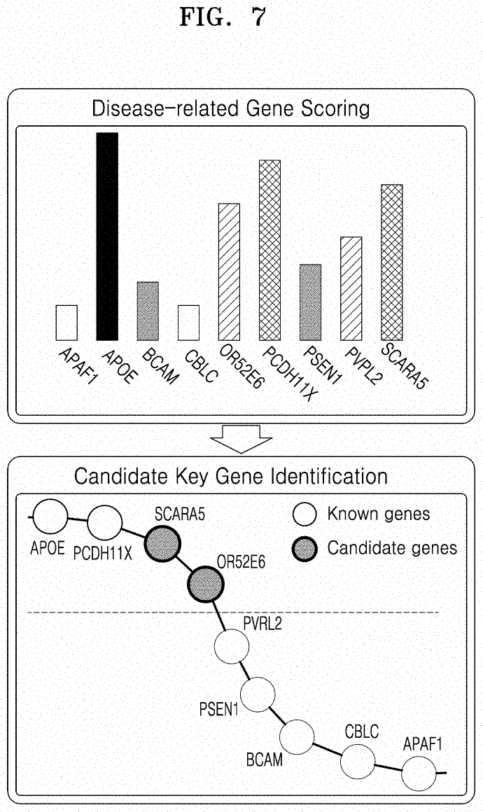

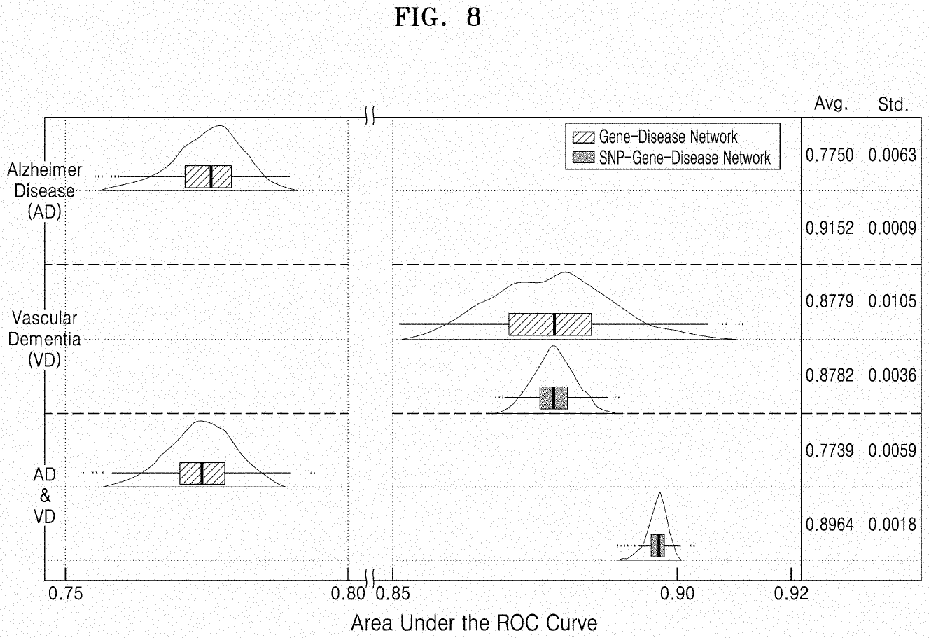

FIGS. 6 and 7 are diagrams for describing operation of identifying candidate genes for genetic diseases using a disease-gene-SNP layered network configured according to an embodiment of the disclosure. FIG. 8 illustrates a candidate gene identification result using the disease-gene-SNP layered network according to an embodiment of the disclosure and a candidate gene identification result using a disease-gene layered network for comparison.

The disease-gene-SNP layered network 100 may have been configured based on data, which is made public in advance, in relation to the genetic disease. In an embodiment of the disclosure, additional candidate genes that may have influence on the genetic disease in addition to the known core genes may be identified by applying the SSL to the configured disease-gene-SNP layered network 100.

In an embodiment of the disclosure, graph-based SSL may be used to identify candidate genes for the genetic disease. The graphic-based SSL may effectively improve classification performance by backing up a classifier based on unlabeled data in an area where there is few labeled data (data with a known target value to be predicted) and plenty of unlabeled data (data without the known target value to be predicted).

The graph-based SSL may build a graph by representing data as nodes and similarity between the nodes as edges, and output predicted values such that similar data points have similar predicted values. n data points may include one labeled data and u unlabeled data.

The graph-based SSL obtains a set of predicted values f that minimizes a target function expressed in the following equation 5 by using the graph Laplacian matrix L.

$${{min}_{f} ( f - y )}^{T} ( f - y ) + μ {f}^{T} Lf \qquad ( 5 )$$

where a label set y may be defined to be y=(y₁, . . . , yₗ, 0, . . . 0)^{T}, in which case a label value of the unlabeled node is equal to 0. The set of predicted values may be defined to be f=(f₁, . . . , fₗ, fₗ₊₁, . . . , Fₙ₌ₗ₊ᵤ)^{T}. Furthermore, the graph Laplacian matrix L is defined to be L=D−W, where d=diag(dᵢ), dᵢ=Σⱼwᵢⱼ, and W refers to a pseudo matrix of a network. Parameter μ is a value set by the user or by default, and a predicted value fᵢ of the labeled node may be set to be similar to the actual value yᵢ of the label set and a predicted value fᵢ of the unlabeled node may be set to be similar to a predicted value fᵢ of a neighboring node.

From the equation 5, a set of predicted values f may be obtained as in the following equation 6. The set of predicted values f may represent predicted values (scores) of n data points (genes), respectively, and I may be an identity matrix of dimension (l+u)×(l+u).

f=(I+μL)⁻¹y (6)

The aforementioned graph-based SSL is usually used for a single network. However, for the disease-gene-SNP layered network 100 according to an embodiment of the disclosure, it may not be easy to apply the equation 6 used for a single network having a complicated structure of three layers.

In an embodiment of the disclosure, the pseudo matrix W of the disease-gene-SNP layered network 100 may be classified into a plurality of intra-matrices W^{{intra}} and a plurality of inter-matrices W^{{inter}}. Based on this, the graph Laplacian matrix L may be represented by a sum of L^{{intra}}(=D^{{intra}}−W^{{intra}}) and L^{{inter}}(=D^{{inter}}−W^{{inter}}.

A final solution for the predicted value may be defined as in the following equation 7:

$$\tilde{f} = {A}^{- 1} Y - {A}^{- 1} {C ( \frac{1}{{μ}_{b}} Q + {C}^{T} {A}^{- 1} C )}^{- 1} {C}^{T} {A}^{- 1} y \qquad ( 7 )$$

where A=I+μₐL^{{intra}}, μₐ and μ_{b} may correspond to smoothing parameters of the intra-layer and inter-layer, respectively. Furthermore, matrices Q and C may be obtained as approximations of the graph Laplacian matrix L^{{inter}} for the inter-matrix as in the following equation 8:

L^{{inter}}≈CQ+C^{T} (8)

where C denotes a sampled column of L^{{inter}}, and Q denotes intersection of C and a corresponding row. The approximated solution may be closer to an actual solution as more rows are sampled. (Kim et al., “Semi-supervised learning for hierarchically structured networks”, Pattern Recognition 95 (2019), 191-200)

The output (predicted value) \tilde{f}. of the equation 7 may have a value ranging from 0 to 1, and may be calculated for all the nodes (genes). After the predicted values are obtained, a score Score(i) of the i-th node (gene) may be calculated as in the following equation 9:

$$Score ( i ) = \frac{1}{1 + {e}^{\text{?}}} \qquad ( 9 )$$

$$\text{?} \text{indicates text missing or illegible when filed}$$

The higher the calculated score, the higher the possibility of being related to the genetic disease, and the gene corresponding to the score may be identified as a candidate gene for the genetic disease.

Referring to FIGS. 6 and 7 together, in the graph-based SSL algorithm, information on the label set y of the equation 7 may be required to identify a candidate gene. First, a label of a node corresponding to a disease (e.g., Alzheimer's disease) to be identified among nodes of the disease network may be set to 1 and the others may be set to 0. In the gene network, a label of nodes corresponding to genes (e.g., ‘APOE’ and ‘PCDH11X’) already known to be related to the disease (e.g., Alzheimer's disease) may be set to 1 and the others may be set to 0.

Furthermore, for the SNP network, a label of SNPs (e.g., ‘rs7412’, ‘rs429358’, and ‘rs769450’) highly associated with the disease (Alzheimer's disease) may be set to 1 and the others may be set to 0. In an embodiment of the disclosure, the SNPs highly associated with the disease may be selected based on at least one test of i) chi-square test, ii) Cochran Armitage trend test, iii) Hardy-Weinberg equilibrium test, iv) Jonckheere-Terpstra test, and v) a logistic regression model. The lowest of the values obtained from the tests may be set to a representative value of the corresponding SNP, and a label of the node corresponding to an SNP for which a set value is lower than a preset reference value may be set to 1.

After the labels are set as described above, the score of each of the genes may be calculated based on the equations 7 to 9 and genes having the score higher than a certain value may be identified as candidate genes for the disease.

In the example of FIGS. 6 and 7, genes already known as core genes related to Alzheimer's disease may correspond to ‘APOE’ and ‘PCDH11X’ according to traditional data. However, in the embodiment of the disclosure where the graph-based SSL is applied to the disease-gene-SNP layered network, it may be determined that ‘SCARA5’ and ‘OR52E6’ genes are identified as candidate genes for Alzheimer's disease in addition to the already known genes. In configuring the disease-gene-SNP layered network 100, the more the data existing for each of the patient group and the control group, the higher the accuracy in identification of the candidate gene.

Referring to FIG. 8, compared to an occasion when the traditional disease-gene layered network is used, performance of identification of candidate genes for a disease affected by a genetic factor, such as the Alzheimer's disease (AD) is significantly improved according to the embodiment of the disclosure. On the other hand, as for vascular dementia (VD), which is affected by external factors such as brain tissue damage caused by the cerebrovascular disease rather than genetic factors, improvement of the identification performance may be relatively low.

According to embodiments of the disclosure, through the disease-gene-SNP layered network configured based on information about intra-relations between SNPs and inter-relations between genes and SNPs, time and cost required to identify candidate genes related to a genetic disease may be efficiently reduced and the identification accuracy may also increase. Accordingly, genetic diseases such as dementia may be detected earlier and prevented.

According to embodiments of the disclosure, candidate genes other than those known to be related to a genetic disease may be effectively identified by using a disease-gene-SNP layered network configured based on information about intra-relations of SNPs and gene-SNP interrelations.

Furthermore, candidate genes related to a genetic disease may be efficiently identified and an accuracy of the identification may also be improved, through a graph based semi-supervised learning (SSL) algorithm optimized for the layered network, even when there is plenty of unlabeled data.

The aforementioned embodiments of the disclosure may be implemented with computer-readable codes on a medium on which a program is recorded. The computer-readable medium is any data storage device that can store data which can be thereafter read by a computer system. For example, the computer-readable medium may include a hard disk drive (HDD), a solid state disk (SSD), a silicon disk drive (SDD), a read-only memory (ROM), a random access memory (RAM), a compact disc ROM (CD-ROM), a magnetic tape, a floppy disk, an optical data storage device, etc.

The aforementioned embodiments are described merely for example with reference to the drawings for more thorough understanding of the disclosure, and should not be construed as limiting the technical idea of the disclosure.

In addition, it will be apparent to those of ordinary skill in the art to which the disclosure pertains that various changes and modifications can be made without departing from basic principles of the disclosure.

What is claimed is:

1. A method of identifying a candidate gene for a genetic disease performed by a computer-executable program, the method comprising:
obtaining a disease network, disease-gene association information, and a gene network;
obtaining a single nucleotide polymorphism (SNP) network based on intra-relation data between a plurality of SNPs, and inter-relation data between genes and SNPs;
creating a disease-gene-SNP layered network based on the disease network, the disease-gene association information, the gene network, the SNP network, and the interrelation data between genes and SNPs; and
identifying a candidate gene for a genetic disease using the layered network.

2. The method of claim 1, wherein the SNP network comprises
a plurality of nodes each corresponding to an SNP type; and
at least one edge representing connection between the plurality of nodes, and
wherein each of the at least one edge represents a degree of similarity based on the intra-relation between the connected SNPs.

3. The method of claim 2, wherein each of the at least one edge has a value obtained based on odds ratios in a logistic regression model based on allele dosage of the connected SNPs.

4. The method of claim 1, wherein the creating of the layered network comprises setting values of gene-SNP edges between the gene network and the SNP network based on the interrelation data between genes and SNPs, and
wherein the setting of the values of gene-SNP edges comprises:
when a first SNP corresponding to a first node among nodes of the SNP network belongs to a first gene corresponding to a second node among nodes of the gene network, setting a value of a gene-SNP edge between the first node and the second node to 1; and
when the first SNP does not belong to the first gene, setting the value of the gene-SNP edge between the first node and the second node to 0.

5. The method of claim 1, wherein the identifying of the candidate gene for the genetic disease using the layered network comprises:
setting a label of nodes corresponding to genes and SNPs already known to be related to the genetic disease among nodes of the layered network to 1 and a label of the other nodes to 0;
calculating a score for each of the genes using graph-based semi-supervised learning (SSL); and
identifying a candidate gene for the genetic disease based on the calculated score.

6. The method of claim 5, wherein the identifying of the candidate gene for the genetic disease based on the calculated score comprises:
identifying at least one gene with the calculated score higher than a reference score as the candidate gene.

7. The method of claim 1, wherein the disease network comprises:
a plurality of nodes each corresponding to a disease; and
at least one edge representing connection between the plurality of nodes,
wherein each of the at least one edge represents a degree of association between the connected diseases, and
wherein the degree of association is calculated based on a number or a rate of genes common between the connected diseases.

8. The method of claim 1, wherein the gene network comprises:
a plurality of nodes each corresponding to a gene; and
at least one edge representing connection between the plurality of nodes,
wherein each of the at least one edge represents a degree of association between the connected genes, and
wherein the degree of association is obtained from a database in which pieces of protein-protein interaction information are integrated.

9. The method of claim 1, wherein the disease-gene association information comprises information about genes related to causing each disease,
wherein the creating of the layered network comprises setting values of disease-gene edges between the disease network and the gene network based on the disease-gene association information, and
wherein the setting of the values of disease-gene edges comprises:
when a first gene corresponding to a first node among nodes of the gene network is identified based on the disease-gene association information as being related to causing a first disease corresponding to a second node among nodes of the disease network, setting a value of a disease-gene edge between the first node and the second node to 1; and
when the first gene is not identified as being related to causing the first disease, setting the value of the disease-gene edge between the first node and the second node to 0.

10. A method of identifying a candidate gene for a genetic disease performed by a computer-executable program, the method comprising:
creating a single nucleotide polymorphism (SNP) network based on intra-relation data between a plurality of SNPs;
creating a gene-SNP layered network based on a gene network, the SNP network, and intra-relation data between SNPs; and
identifying a candidate gene for a genetic disease using the layered network.

11. The method of claim 10, wherein the SNP network comprises
a plurality of nodes each corresponding to an SNP type; and
at least one edge representing connection between the plurality of nodes,
wherein each of the at least one edge has a value based on the intra-relation between the connected SNPs.

12. The method of claim 10, wherein the creating of the gene-SNP layered network comprises setting values of gene-SNP edges between the gene network and the SNP network based on the interrelation data between genes and SNPs, and
wherein the setting of the values of gene-SNP edges comprises:
when a first SNP corresponding to a first node among nodes of the SNP network belongs to a first gene corresponding to a second node among nodes of the gene network, setting a value of a gene-SNP edge between the first node and the second node to 1; and
when the first SNP does not belong to the first gene, setting the value of the gene-SNP edge between the first node and the second node to 0.

13. The method of claim 10, wherein the identifying of the candidate gene comprises:
creating a disease-gene-SNP layered network based on the gene-SNP layered network, a disease network, and disease-gene association information; and
identifying a candidate gene for a genetic disease using the disease-gene-SNP layered network.

14. The method of claim 13, wherein the identifying of the candidate gene for a genetic disease using the disease-gene-SNP layered network comprises:
setting a label of nodes corresponding to genes and SNPs already known to be related to the genetic disease among nodes of the disease-gene-SNP layered network to 1 and a label of the other nodes to 0;
calculating a score for each of the genes using graph-based semi-supervised learning (SSL); and
identifying a candidate gene for the genetic disease based on the calculated score.

15. The method of claim 14, wherein the identifying of the candidate gene for the genetic disease based on the calculated score comprises
identifying at least one gene with the calculated score higher than a reference score as the candidate gene.
